(19) United States
(12) Patent Application Publication
(10) Pub. No.: US 2025/0304527 A1
(43) Pub. Date: Oct. 2, 2025

(54) PROCESS FOR MAKING ETHANOLAMINES, POLYETHYLENIMINE AND AMMONIA BASED ON NON-FOSSIL ENERGY

(71) Applicant: BASF SE, Ludwigshafen am Rhein (DE)

(72) Inventors: Stephan HUEFFER, Ludwigshafen am Rhein (DE); Marco KRUEGER, Ludwigshafen am Rhein (DE); Thomas WEISS, Ludwigshafen am Rhein (DE)

(21) Appl. No.: 18/862,181

(22) PCT Filed: Apr. 28, 2023
(86) PCT No.: PCT/EP2023/061271

§ 371 Date: Nov. 1, 2024

(30) Foreign Application Priority Data
May 2, 2022 (EP) 22171101.3

(51) Int. Cl. C07C 213/04; C01C 1/04; C07C 29/151; C07C 29/44; C25B 1/04

(52) U.S. Cl. CPC C07C 213/04; C01C 1/0405; C07C 29/151; C07C 29/44; C25B 1/04

(57) ABSTRACT

Ethanolamines, polyethylenimine and ammonia having a low molar share of deuterium, a process for making ethanolamines, polyethylenimine and ammonia based on non-fossil energy, the use of the molar share of deuterium in hydrogen and downstream compounds based on hydrogen for tracing the origin of preparation of hydrogen and downstream compounds based on hydrogen, and a process for tracing the origin of preparation of hydrogen and downstream compounds based on hydrogen by determining the molar share of deuterium in hydrogen and said downstream compounds based on hydrogen, applications of the polyethylenimine and the use of the polyethylenimine, and the use of the ethanolamines, preferably monoethanolamine and/or diethanolamine, or the polyethylenimine as liquid or solid CO₂ absorbents in CO₂ capturing processes.

The present invention relates to ethanolamines, selected from monoethanolamine, diethanolamine, triethanolamine and mixtures thereof, polyethylenimine and ammonia having a low molar share of deuterium, a process for making ethanolamines, selected from monoethanolamine, diethanolamine, triethanolamine and mixtures thereof, polyethylenimine and ammonia based on non-fossil energy, the use of the molar share of deuterium in hydrogen and downstream compounds based on hydrogen for tracing the origin, especially the energetic origin, of the hydrogen and downstream compounds based on hydrogen, wherein the compounds are preferably ethanolamines, selected from monoethanolamine, diethanolamine, triethanolamine and mixtures thereof, polyethylenimine or ammonia, and a process for tracing the origin, especially the energetic origin, of hydrogen and downstream compounds based on hydrogen by determining the molar share of deuterium in hydrogen and said downstream compounds based on hydrogen, wherein the compounds are preferably ethanolamines, selected from monoethanolamine, diethanolamine, triethanolamine and mixtures thereof, polyethylenimine or ammonia, applications of the polyethylenimine and the use of the polyethylenimine, and the use of the ethanolamines, preferably monoethanolamine and/or diethanolamine, or the polyethylenimine as liquid or solid CO₂ absorbents in CO₂ capturing processes.

Ethanolamines are flammable, corrosive, colorless, viscous liquids that are produced by the reaction between ammonia and ethylene oxide (EO). Identified many years ago, ethanolamines are a key ingredient in a number of important product formulations such as cosmetics and personal hygiene applications, agricultural products, woodpreservation chemicals, soaps and detergents, gas treatments. They can also be used in the production of nonionic detergents, emulsifiers, and soaps, as well as in emulsion paints, polishes, and cleansers. There are 3 types of ethanolamines: monoethanolamines (MEA), diethanolamines (DEA), and triethanolamines (TEA). The formation of MEA, DEA, or TEA depends on whether an ammonia molecule reacts with one, two, or three ethylene oxide molecules.

Polyethylenimine (PEI) is a versatile polymer that can be used for various purposes such as, but not limited to detergents, adhesives, water treatment agents and cosmetics. Furthermore, PEI is used in paper manufacture as well as in flocculating processes or as a raw material in the field of biotechnology.

Further, PEI and well as MEA and/or DEA are useful as capturing agent for carbon dioxide (CO₂). The amino groups in PEI react with the CO₂.

Ammonia is a key precursor in the preparation of MEA, which is in turn a key precursor in the preparation of PEI.

Since the development of the Haber-Bosch process for the preparation of ammonia, the vast majority of ammonia is manufactured by the direct synthesis from hydrogen and nitrogen in the presence of a catalyst, especially an iron-containing catalyst. Special care needs to be taken with the provision of the starting materials hydrogen and nitrogen. They should exhibit a high purity and be substantially free from catalyst poisoning agents such as carbon monoxide and sulfur compounds such as H₂S and SO₂. In modern processes, a significant amount of the hydrogen is provided by steam reforming, thus, from natural gas.

However, the petrochemical steam reforming process has its negative impacts with regard to its carbon footprint including the consumption of a lot of fossil-based natural resources and energy.

US 2011/136097 relates to a method for determining origins of food products, more specifically for determining the geographic and/or biological origin of food products containing alcohols or sugars by using the specific isotope ratios of for example sugars from different plants, which is influenced by climate conditions and the area of origin as isotopic “fingerprint” of the specific plants.

However, the deuterium content taken advantage of in the present invention is not the natural “fingerprint”, but the finding that the deuterium content of hydrogen obtained by electrolysis of water is lower than the naturally occurring deuterium content of hydrogen. Further, not the geographic area of origin is determined, but the preparation process of the hydrogen.

U.S. Pat. No. 6,495,609 concerns a method for recovering carbon dioxide from an ethylene oxide production process and using the recovered carbon dioxide as a carbon source for methanol synthesis. However, the hydrogen used in the process of U.S. Pat. No. 6,495,609 is present in syngas, such as natural gas or refinery off-gas.

GB 2 464 691 A relates to the manufacture of methanol from agricultural by-product cellulosic/lignitic material. In a first section of a synthesis factory, the cellulosic/lignitic by-product that remains after the cropping of agricultural products is converted to carbon dioxide by calorific oxidation. In another section of a synthesis factory, hydrogen gas is produced by electrolysis which is then reacted with carbon dioxide to make methanol.

WO 2016/149507 A1 relates to the oxidative coupling of methane for obtaining a high number of different products. Claim 217 for example discloses a method for producing oxalate compounds.

U.S. Pat. No. 7,119,231 B2 relates to a process for preparing alkanolamines by reacting ammonia with alkylene oxide in a reaction space in the presence of a catalyst to give monoalkanolamine or dialkanolamine or trialkanolamine or a mixture of two or three of these compounds. There is no hint concerning the deuterium content of the hydrogen comprising compounds employed in U.S. Pat. No. 7,119,231 B2 or concerning the use of non-fossil energies.

FR 2 851 564 A1 concerns a process for preparation of ethylene oxide and ethanolamines. As in FR 2 851 564 A1 does not contain any hint to the presence of deuterium in the hydrogen-comprising compounds or the use of non-fossil energies.

US 2008/0283411 A1 relates to a method for converting a carbon source and a hydrogen source into hydrocarbons. It is mentioned that the method and the device are useful to produce a fossil fuel alternative energy source, store renewable energy, sequester carbon dioxide from the atmosphere, counteract global warming, and store carbon dioxide in a liquid fuel.

WO 2015/102985 A1 relates to a process for the preparation of ethanolamines comprising reacting a water-ammonia solution with ethylene oxide. However, there is no hint in WO 2015/102985 A1 concerning the preparation of hydrogen by electrolysis, the use of renewable energies and the presence of deuterium in the hydrogen-containing compounds disclosed in WO 2015/102985 A1.

DE 195 34 493 A1 relates to a process for the preparation of aziridines in the presence of fine-particle shell catalysts. The aziridine is prepared by dehydration of alkanolamine in the presence of said catalysts. However, neither an electrolysis of water for the preparation of hydrogen nor the deuterium content of the hydrogen-containing compounds mentioned in DE 195 34 493 A1 nor the use of renewable energies is mentioned in DE 195 34 493 A1.

It is therefore an object of the present invention to provide environmentally friendly ethanolamines, selected from monoethanolamine, diethanolamine, triethanolamine and mixtures thereof, polyethylenimine and ammonia and an environmentally friendly process for making the same, that process uses as little fossil-based energy as possible.

The object is achieved by ethanolamines, selected from monoethanolamine, diethanolamine, triethanolamine and mixtures thereof, wherein the molar share of deuterium is ≤100 ppm, preferably in the range of from 10 to ≤98 ppm, more preferably in the range of from 10 to ≤95 ppm, most preferably in the range of from 10 to ≤90 ppm, based on the total hydrogen content; polyethylenimine having a molar share of deuterium of ≤110 ppm, preferably in the range of from 10 to ≤105 ppm, more preferably in the range of from 10 to ≤95 ppm, most preferably in the range of from 10 to ≤92 ppm, based on the total hydrogen content; and ammonia wherein the molar share of deuterium is ≤100 ppm, preferably in the range of from 10 to ≤95 ppm, more preferably in the range of from 10 to ≤90 ppm, most preferably in the range of from 10 to ≤80 ppm, based on the total hydrogen content.

In a further embodiment of the present invention, the object is achieved by a process for making ethanolamines, selected from monoethanolamine, diethanolamine, triethanolamine and mixtures thereof, wherein said process comprises the following steps:
(a) providing hydrogen with a molar share of deuterium ≤100 ppm, preferably in the range of from 10 to ≤95 ppm, more preferably in the range of from 10 to ≤90 ppm, most preferably in the range of from 10 to ≤80 ppm, based on the total hydrogen content, by electrolysis based on electrical power generated at least in part from non-fossil energy,
(b) reacting the hydrogen from step (a) with nitrogen to form ammonia,
(c) reacting the hydrogen from step (a) with carbon oxides, preferably carbon dioxide to form methanol,
(d) converting the methanol from step (c) to ethylene and further to ethylene oxide, (e) converting the ammonia from step (b) with ethylene oxide from step (d) to ethanolamines in one or more steps.

The object is further achieved by a process for preparing polyethylenimine, wherein said process comprises:
(f) separating monoethanolamine from ethanolamines obtained in steps (a) to (e) of the process according to any one of claims 1 to 7, or from ethanolamines according to claim 8;
(g) converting monoethanolamine to ethylenimine,
(h) polymerizing ethylenimine from step (g) to polyethylenimine; and
by a process for making ammonia wherein said process comprises the following steps:
(a) providing hydrogen with a molar share of deuterium ≤100 ppm, preferably in the range of from 10 to ≤95 ppm, more preferably in the range of from 10 to ≤90 ppm, most preferably in the range of from 10 to ≤80 ppm, based on the total hydrogen content, by electrolysis based on electrical power generated at least in part from non-fossil energy,
(b) reacting the hydrogen from step (a) with nitrogen to form ammonia.

Further, it is important that the origin of the hydrogen and downstream compounds obtained by clean energy can be tracked in a reliable way.

This is especially in order to ensure that:
Hydrogen and downstream compounds have been produced in accordance with sustainability criteria.
Renewable attributes aren't subject to double counting.

Companies are placing increasing importance on sourcing green energy. Because of this, tracking systems have to be developed for the origin of the energy used in the preparation of hydrogen and downstream compounds.

This object is also achieved by use of the molar share of deuterium in hydrogen and downstream compounds based on hydrogen for tracing the origin, especially the energetic origin, of the hydrogen and downstream compounds based on hydrogen, wherein the compounds are preferably ethanolamines, selected from monoethanolamine, diethanolamine, triethanolamine and mixtures thereof, polyethylenimine or ammonia and a process for tracing the origin, especially the energetic origin, of hydrogen and downstream compounds based on hydrogen by determining the molar share of deuterium in hydrogen and said downstream compounds based on hydrogen, wherein the compounds are preferably ethanolamines, selected from monoethanolamine, diethanolamine, triethanolamine and mixtures thereof, polyethylenimine or ammonia. Methods for determination of the molar share of deuterium in hydrogen and downstream compounds based on hydrogen are known to a person skilled in the art. A suitable method is described in the examples of the present application.

A further environmental benefit of the environmentally friendly ethanolamines, selected from monoethanolamine, diethanolamine, triethanolamine and mixtures thereof and polyethylenimine according to the present invention is their use in carbon capturing processes, since the ethanolamines, selected from monoethanolamine, diethanolamine, triethanolamine and mixtures thereof and polyethylenimine according to the present invention are produced using as little fossil-based energy as possible, ideally no fossil-based energy, and do therefore only add as little as possible, ideally nothing, to CO₂ emission.

A further embodiment of the present invention is therefore the use of the ethanolamines, selected from monoethanolamine, diethanolamine, triethanolamine and mixtures thereof, or the polyethylenimine according to the present invention as liquid or solid CO₂ absorbents in CO₂ capturing processes.

The molar share of deuterium in hydrogen and downstream compounds based on hydrogen is given in the present application in ppm, based on the total hydrogen content, which is the mol-ppm content of deuterium, based on the total hydrogen content (in hydrogen or in the compounds discussed, respectively).

The deuterium content of hydrogen and downstream compounds based on hydrogen is given in the present application in atom-ppm based on the total molar hydrogen content (total atoms of protium ¹H and deuterium ²H). The terms “deuterium content” and “molar share of deuterium” are used synonymously throughout the application.

In physical organic chemistry, a kinetic isotope effect is the change in the reaction rate of a chemical reaction when one of the atoms in the reactants is replaced by one of its isotopes. Formally, it is the ratio of rate constants k_{L}/k_{H} for the reactions involving the light (k_{L}) and the heavy (k_{H}) isotopically substituted reactants (isotopologues). This change in reaction rate is a quantum mechanical effect that primarily results from heavier isotopologues having lower vibrational frequencies compared to their lighter counterparts. In most cases, this implies a greater energetic input needed for heavier isotopologues to reach the transition state, and consequently, a slower reaction rate.

Isotopic rate changes are most pronounced when the relative mass change is greatest, since the effect is related to vibrational frequencies of the affected bonds. For instance, changing a hydrogen atom (H) to its isotope deuterium (D) represents a 100% increase in mass, whereas in replacing ¹²C with ¹³C, the mass increases by only 8 percent. The rate of a reaction involving a C—H bond is typically 6-10 times faster than the corresponding C-D bond, whereas a ¹²C reaction is only 4 percent faster than the corresponding ¹³C reaction.

A primary kinetic isotope effect may be found when a bond to the isotope atom is being formed or broken. A secondary kinetic isotope effect is observed when no bond to the isotope atom in the reactant is broken or formed. Secondary kinetic isotope effects tend to be much smaller than primary kinetic isotope effects; however, secondary deuterium isotope effects can be as large as 1.4 per deuterium atom.

Process for making ethanolamines comprising steps (a) to (e) as mentioned above:

### Step (a)

Step (a) concerns the provision of hydrogen with a molar share of deuterium below 90 ppm, based on the total hydrogen content, by electrolysis based on electrical power generated at least in part from non-fossil energy.

The electrical power is generated at least in part from non-fossil resources.

The term “at least in part” means that part of the electrical power can still be produced from fossil fuels, preferably from natural gas, since combustion of natural gas causes much lower carbon dioxide emission per Megajoule of electrical energy produced than combustion of coal. However, the portion of electrical energy produced from fossil fuels should be as low as possible, preferably ≤50%, preferably ≤30%, most preferably ≤20%, further most preferably ≤10%. In one embodiment, the electrical power is generated exclusively from non-fossil resources.

Various methods for certification and tracking of the “energy source mix” have been set up based on local legislations. Certificates such as “Non-Fossil Certificate Contracts” are common practice for tracking the ratio of non-fossil energy used in industrial processes and related products (https://www.ekoenergy.org/ecolabel/criteria/tracking/)

Preferably, the electrical power is generated at least in part from wind power, solar energy (thermal, photovoltaic and concentrated solar power), hydroelectricity (tidal power, wave power, hydroelectric dams, In-river-hydrokinetics), geothermal energy, ambient or industrial heat captured by heat pumps, bioenergy (biofuel, biomass), the renewable part of waste energy sources or nuclear energy (fission).

In a further embodiment, the electrical power is generated at least in part from renewable resources, preferably from wind power, solar energy (thermal, photovoltaic and concentrated solar power), hydroelectricity (tidal power, wave power, hydroelectric dams, In-river-hydrokinetics), geothermal energy, ambient heat captured by heat pumps, bioenergy (biofuel, biomass), or the renewable part of waste.

The types of electrical power resources mentioned above are generally known by a person skilled in the art.

In one preferred embodiment of the inventive process, the electrical power from non-fossil resources used in the electrolysis according to the invention can be generated at least in part by nuclear energy. The nuclear energy can be obtained by fission. Fission occurs when a neutron enters a larger atomic nucleus, forcing it to excite and spilt into two smaller atoms—also known as fission products. Additional neutrons are also released that can initiate a chain reaction. When each atom splits, a tremendous amount of energy is released. Uranium and plutonium isotopes are most commonly used for fission reactions in nuclear power reactors because they are easy to initiate and control. The energy released by fission in these reactors heats water into steam. The steam is used to spin a turbine to produce carbon-free electricity.

In one preferred embodiment of the inventive process, the electrical power used in electrolysis is generated at least in part from wind power. Wind power can be used to run wind turbines. Modern utility-scale wind turbines range from around 600 kW to 9 MW of rated power. The power available from the wind is a function of the cube of the wind speed, so as wind speed increases, power output increases up to the maximum output for the particular turbine. Areas where winds are stronger and more constant, such as offshore and high-altitude sites, are preferred locations for wind farms.

In one further preferred embodiment of the inventive process, the electrical power used in electrolysis is generated at least in part from solar power, particularly preferred from photovoltaic systems. A photovoltaic system converts light into electrical direct current (DC) by taking advantage of the photoelectric effect. Concentrated solar power (CSP) systems use lenses or mirrors and tracking systems to focus a large area of sunlight into a small beam. CSP-Stirling currently has by far the highest efficiency among all solar energy technologies.

In one preferred embodiment of the inventive process, the electrical power used in electrolysis is generated at least in part from hydropower. There are many forms of hydropower. Traditionally, hydroelectric power comes from constructing large hydroelectric dams and reservoirs. Small hydro systems are hydroelectric power installations that typically produce up to 50 MW of power. They are often used on small rivers or as a low-impact development on larger rivers. Run-of-the-river hydroelectricity plants derive energy from rivers without the creation of a large reservoir. The water is typically conveyed along the side of the river valley (using channels, pipes and/or tunnels) until it is high above the valley floor, whereupon it can be allowed to fall through a penstock to drive a turbine.

Wave power, which captures the energy of ocean surface waves, and tidal power, converting the energy of tides, are two forms of hydropower with future potential.

In one further preferred embodiment of the inventive process, the electrical power used in electrolysis is generated at least in part from geothermal energy. Geothermal energy is the heat that comes from the sub-surface of the earth. It is contained in the rocks and fluids beneath the earth's crust and can be found as far down to the earth's hot molten rock, magma.

To produce power from geothermal energy, wells are dug a mile deep into underground reservoirs to access the steam and hot water there, which can then be used to drive turbines connected to electricity generators. There are three types of geothermal power plants; dry steam, flash and binary.

Dry steam is the oldest form of geothermal technology and takes steam out of the ground and uses it to directly drive a turbine. Flash plants use high-pressure hot water into cool, low-pressure water whilst binary plants pass hot water through a secondary liquid with a lower boiling point, which turns to vapor to drive the turbine.

In one further preferred embodiment of the inventive process, the electrical power used in electrolysis is generated at least in part from biomass. Biomass is biological material derived from living, or recently living organisms. It most often refers to plants or plantderived materials which are specifically called lignocellulosic biomass. As an energy source, biomass can either be used directly via combustion to produce heat (e.g. heat from fermentation processes) or electricity, or indirectly after converting it to various forms of biofuel and gas. Conversion of biomass to biofuel can be achieved by different methods which are broadly classified into: thermal, chemical, and biochemical methods. Wood was the largest biomass energy source as of 2012; examples include forest residues—such as dead trees, branches and tree stumps—, yard clippings, wood chips and even municipal solid waste. Industrial biomass can be grown from numerous types of plants, including miscanthus, switchgrass, hemp, corn, poplar, willow, sorghum, sugarcane, bamboo, and a variety of tree species, ranging from eucalyptus to oil palm (palm oil).

Plant energy is produced by crops specifically grown for use as fuel that offer high biomass output per hectare with low input energy. The grain can be used for liquid transportation fuels while the straw can be burned to produce heat or electricity. Biomass can be converted to other usable forms of energy such as methane gas or transportation fuels such as ethanol and biodiesel. Rotting garbage, and agricultural and human waste, all release methane gas—also called landfill gas or biogas. Crops, such as corn and sugarcane, can be fermented to produce the transportation fuel, ethanol. Biodiesel, another transportation fuel, can be produced from left-over food products such as vegetable oils and animal fats.

Biopower technologies convert renewable biomass fuels into heat and electricity using processes like those used with fossil fuels. There are three ways to harvest the energy stored in biomass to produce biopower: burning, bacterial decay, and conversion to a gas or liquid fuel. Biopower can offset the need for carbon fuels burned in power plants, thus lowering the carbon intensity of electricity generation. Unlike some forms of intermittent renewable energy, biopower can increase the flexibility of electricity generation and enhance the reliability of the electric grid.

The electrolysis in step (a) is generally an electrolysis of water.

Electrolysis of water is an environmentally friendly method for production of hydrogen because it uses renewable H₂O and produces only pure oxygen as by-product.

According to the present invention, the electrolysis which is generally a water electrolysis utilizes as electrical power direct current (DC) at least in part from non-fossil energy resources.

It is now observed as a key observation of the present application that by the electrolysis of water, the deuterium atom content of the hydrogen is lower than in the hydrogen generated petrochemically, for example as contained in fossil-based synthesis gas, i.e. ≤100 ppm, preferably in the range of from 10 to ≤95 ppm, more preferably in the range of from 10 to ≤90 ppm, most preferably in the range of from 10 to ≤80 ppm, based on the total hydrogen content. The deuterium atom content in electrolytically produced hydrogen may be as low as 10 ppm. The remaining deuterium is mainly present in the form of D-H rather than D₂.

One suitable water electrolysis process is alkaline water electrolysis. Hydrogen production by alkaline water electrolysis is a well-established technology up to the megawatt range for a commercial level. In alkaline water electrolysis initially at the cathode side two water molecules of alkaline solution (KOH/NaOH) are reduced to one molecule of hydrogen (H₂) and two hydroxyl ions (OH⁻). The produced H₂ emanates from the cathode surface in gaseous form and the hydroxyl ions (OH⁻) migrate under the influence of the electrical field between anode and cathode through the porous diaphragm to the anode, where they are discharged to half a molecule of oxygen (O₂) and one molecule of water (H₂O). Alkaline electrolysis operates at lower temperatures such as 30-100° C. with alkaline aqueous solution (KOH/NaOH) as the electrolyte, the concentration of the electrolyte being about 20% to 30%. The diaphragm in the center of the electrolysis cell separates the cathode and anode and also separates the produced gases from their respective electrodes, avoiding the mixing of the produced gases.

An overview of hydrogen production by alkaline water electrolysis powered by renewable energy is given in J. Brauns and T. Turek in Processes, 8(2) (2020), pp. 248.

In one further embodiment of the inventive process, hydrogen is provided by polymer electrolyte membrane water electrolysis. Variants of polymer electrolyte membrane water electrolysis are proton exchange membrane water electrolysis (PEMWE, PEM water electrolysis) and anion exchange membrane water electrolysis (AEMWE, AEM water electrolysis).

PEM water electrolysis technology is similar to the PEM fuel cell technology, where solid polysulfonated membranes (Nafion®, fumapem®) are used as an electrolyte (proton conductor). These proton exchange membranes have many advantages such as low gas permeability, high proton conductivity (0.1±0.02 S cm⁻¹), low thickness (20-300 μm), and allow high-pressure operation. In terms of sustainability and environmental impact, PEM water electrolysis is one of the most favorable methods for conversion of renewable energy to highly pure hydrogen. PEM water electrolysis has great advantages such as compact design, high current density (above 2 A cm⁻²), high efficiency, fast response, operation at low temperatures (20-90° C.) and production of ultrapure hydrogen. The state-of-the-art electrocatalysts for PEM water electrolysis are highly active noble metals such as Pt/Pd for the hydrogen evolution reaction (HER) at the cathode and IrO₂/RuO₂ for the oxygen evolution reaction (OER) at the anode.

One of the largest advantages of PEM water electrolysis is its ability to operate at high current densities. This can result in reduced operational costs, especially for systems coupled with very dynamic energy sources such as wind and solar power, where sudden spikes in energy output would otherwise result in uncaptured energy. The polymer electrolyte allows the PEM water electrolyzer to operate with a very thin membrane (ca. 100-200 μm) while still allowing high operation pressure, resulting in low ohmic losses, primarily caused by the conduction of protons across the membrane (0.1 S/cm), and a compressed hydrogen output.

The PEM water electrolyzer utilizes a solid polymer electrolyte (SPE) to conduct protons from the anode to the cathode while insulating the electrodes electrically. Under standard conditions the enthalpy required for the formation of water is 285.9 kJ/mol. One portion of the required energy for a sustained electrolysis reaction is supplied by thermal energy and the remainder is supplied through electrical energy.

The half reaction taking place on the anode side of a PEM water electrolyzer is commonly referred to as the Oxygen Evolution Reaction (OER). Here the liquid water reactant is supplied to a catalyst where it is oxidized to oxygen, protons and electrons.

The half reaction taking place on the cathode side of a PEM water electrolyzer is commonly referred to as the Hydrogen Evolution Reaction (HER). Here the protons that have moved through the membrane are reduced to gaseous hydrogen.

PEMs can be made from either pure polymer membranes or from composite membranes, where other materials are embedded in a polymer matrix. One of the most common and commercially available PEM materials is the fluoropolymer PFSA (e.g. Nafion®, a DuPont product). While Nafion® is an ionomer with a perfluorinated backbone like Teflon, there are many other structural motifs used to make ionomers for proton-exchange membranes. Many use polyaromatic polymers, while others use partially fluorinated polymers.

An overview over hydrogen production by PEM water electrolysis is given in S. Kumar and V. Himabindu, Material Science for Energy Technologies 2 (2019), pp. 4442 4454.

The AEM water electrolysis technology adopts low-cost catalytic materials, as in alkaline electrolysis, and a solid polymer electrolyte architecture, as in PEM electrolysis technology. AEM electrolysis technology operates in an alkaline environment (pH˜10), making it possible the use modest non-noble-metal electrocatalysts (i.e. platin group metal free catalysts=PGM free catalysts), whilst accommodating a zero-gap architecture. The membrane used in this type of electrolysis is a polymeric membrane, containing quaternary ammonium salts. It is relatively inexpensive and has low interaction with atmospheric CO₂.

### Catalysts:

As examples for hydrogen evolution reaction (HER) catalysts, catalysts based on Ni—Mo alloyed materials are suitable.

As examples for oxygen evolution reaction (OER) catalysts, high activity of transition metal mixed oxides are suitable. Specific examples are CuxCo3_xO4, NiCo2O4:Fe and Ni—Fe alloys on Ni foam supports, for example the PGM-free catalysts (Ni—Fe, Ni—Mo, Ni/(CeO2-La2O3)/C and CuxCo3_xO4).

### Membranes and Ionomers:

The chemical stability of AEMs under alkaline conditions has improved markedly due to the development of stabilized functional groups on the polymer backbone. This allows the use of such membranes in AEM electrolysis at higher temperatures for long periods. Suitable membranes and ionomers are known by a person skilled in the art and for example described in the review mentioned below. One example is the commercial membrane Tokuyama A201.

Membrane electrode assembly preparation and cell performance:

The physical and electrochemical characterization of the membrane electrode assembly prepared by either the catalyst-coated substrate (CCS) or the catalyst-coated membrane (CCM) method suggests that the CCM is preferable because improvements in ionic conductivity far outweigh any improvements in electronic conductivity. Liquid electrolyte: Pure water feeds generally result in poor current densities while 1% K₂CO₃ or dilute KOH solutions give good results. A good electrolysis performance is achieved with a 1% K₂CO₃ electrolyte. It is therefore preferable that the water electrolyte comprises 0.1 to 2 wt % K₂CO₃ or KOH.

An overview over hydrogen production by anion exchange membrane water electrolysis is given in H. A. Miller et al., Sustainable Energy Fuels, 2020, 4, pp. 2114-2133.

Beside the alkaline water electrolysis, the AEM and PEM, a further commercially available electrolysis technology is the solid oxide electrolysis (SOE).

SOEC (solid oxide electrolysis cell) feeds water into the cathode and the water undergoes water reduction reaction (WRR), which converts water into hydrogen gas and oxide ions. This hydrogen gas is later brought to purification modules to separate hydrogen gas from the remaining water. Then, the oxide ions migrate from cathode to anode and they release electrons to external circuit to become oxygen gas via oxygen evolution reaction (OER). Typically, the operating temperatures for SOFCs are from 800 to 1,000° C., because high temperatures are required to thermally activate the migration of oxide ions and to facilitate electrochemical reactions on both electrodes. As a result, the overall efficiency is improved. The SOEC is for example described in K. Kamlungsua et al., FUEL CELLS 20, 2020, No. 6, 644-649.

Preferably, the electrolysis in step (a) is a water electrolysis, more preferably PEM water electrolysis, alkaline water electrolysis, or AEM water electrolysis.

In a further preferred embodiment, the electrolysis in step (a) is a solid oxide water electrolysis (SOE).

It is known in the art that deuterium in the evolving hydrogen gas can be depleted with regard to feed water in water electrolysis, e.g. polymer electrolyte membrane water electrolysis. The depletion factor is depending on the electrolysis conditions (water flow, current density). Since the average deuterium content (molar share of deuterium) of water is about 150 ppm, based on the total hydrogen content, hydrogen provided in step (a) of the inventive process has a molar share of deuterium (deuterium content) of ≤100 ppm, preferably in the range of from 10 to ≤95 ppm, more preferably in the range of from 10 to ≤90 ppm, most preferably in the range of from 10 to ≤80 ppm, based on the total hydrogen content, or even lower.

Generally, any water source can be used in the preferred water electrolysis in step (a). However, since the hydrogen prepared in step (a) has a molar share of deuterium (deuterium content) below ≤100 ppm, preferably in the range of from 10 to ≤95 ppm, more preferably in the range of from 10 to ≤90 ppm, most preferably in the range of from 10 to ≤80 ppm, based on the total hydrogen content, it is preferable to use water having a molar share of deuterium (deuterium content) below 160 ppm, based on the total hydrogen content.

Vienna Standard Mean Ocean Water (VSMOW) is an isotopic water standard defined in 1968 by the International Atomic Energy Agency. Despite the somewhat misleading phrase “ocean water”, VSMOW refers to pure water (H₂O) and does not include any salt or other substances usually found in seawater. VSMOW serves as a reference standard for comparing hydrogen and oxygen isotope ratios, mostly in water samples. Very pure, distilled VSMOW water is also used for making high accuracy measurement of water's physical properties and for defining laboratory standards since it is considered to be representative of “average ocean water”, in effect representing the water content of Earth.

The isotopic composition of VSMOW water is specified as ratios of the molar abundance of the rare isotope in question divided by that of its most common isotope and is expressed as parts per million (ppm). For instance ¹⁶O (the most common isotope of oxygen with eight protons and eight neutrons) is roughly 2,632 times more prevalent in sea water than is ¹⁷O (with an additional neutron). The isotopic ratios of VSMOW water are defined as follows:
²H/¹H=155.76±0.1 ppm (a ratio of 1 part per approximately 6420 parts)
³H/¹H=1.85±0.36 &times; 10⁻¹¹ ppm (a ratio of 1 part per approximately 5.41 &times;
10¹⁶ parts, ignored for physical properties-related work)
¹⁸O/¹⁶O=2005.20±0.43 ppm (a ratio of 1 part per approximately 498.7 parts)
¹⁷O/¹⁶O=379.9±1.6 ppm (a ratio of 1 part per approximately 2632 parts)
(see: https://en-academic.com/dic.nsf/enwiki/753132)

More preferably, the water in step (a) has an average deuterium content of 1 ppm (super light water to 156 ppm, based on the total hydrogen content, most preferably 2 ppm to 150 ppm, based on the total hydrogen content.

Processes for the depletion of deuterium in water are known by a person skilled in the art. However, said processes are generally energy consuming electrolysis processes as e.g. described in CN103848399A.

In the case that deuterium depleted water is used, it is therefore preferred to employ deuterium depleted water obtained from the following resources:
A byproduct of “heavy water” (D₂O) production (heavy water has applications in organic chemistry, drug development, and nuclear reactors); (deuterium content about 10-120 ppm)
High mountain water; (deuterium content about 120-150 ppm)
Surface river and lake water; (deuterium content about 130-150 ppm)
Any water source with seasonally low deuterium content e.g. water collected at low temperature (cold winter water contains less deuterium than warm summer water); e.g. water obtained in winter time, e.g. from snow or ice; (deuterium content about 120-150 ppm)
Pole water and antarctic glacier water (deuterium content about 90-150 ppm)
Low salinity sea water e.g. close to river mouths, desalinated sea water or brackish water and waste water treatment effluent water; (deuterium content about 130-155 ppm)

### Step (b)

Step (b) concerns reacting the hydrogen from step (a) with nitrogen to form ammonia,

The reaction of step (b) preferably follows the Haber-Bosch process.

The catalysts usually used in the Haber-Bosch process generally fall into one of two categories, fused-iron and supported metallic catalysts. Fused-iron catalysts are derived from iron oxides, of which there are three possibilities: Fe₂O₃, Fe₃O₄, and Fe₁₋ₓO, which are known as hematite, magnetite, and wüstite, respectively. Industrially, these iron catalysts will be multipromoted with promoters such as K₂O, BaO, KOH, CaO, MgO and Al₂O₃ are present in small quantities of a few weight percentage.

Supported metallic catalysts are catalysts made up of a metallic catalyst material, normally ruthenium or cobalt for the ammonia synthesis reaction, present on the surface of a support material, normally activated carbon or a metal oxide. Commonly the weight percentage of the metallic catalyst is around 2-10%.

Other catalysts which may be used are nickel, and nitride catalyst systems or electride, hydride, nitride, oxynitride hydride promoted Ru, Fe, Co, and Ni catalysts.

The catalysts mentioned above can be used for both conventional centralized large-scale Haber-Bosch ammonia synthesis plants and distributed small-scale ammonia production via the same process.

In one embodiment of the present invention, step (b) is performed at a pressure in the range of from 50 to 350 bar (abs), preferably 150 to 300 bar (abs).

In one embodiment of the present invention, step (b) is performed at a temperature in the range of from 300 to 600° C., preferably 400 to 500° C.

The overall kinetic isotope effect is cumulative, since it will also be present in all subsequent production steps downstream the value chain. By performing step (b), ammonia is formed. The deuterium content is even lower than corresponding to the distribution obtained by classical petrochemical routes.

### Step (c)

Step (c) concerns reacting the hydrogen from step (a) with carbon oxides, preferably carbon dioxide to form methanol.

Suitable carbon oxides are carbon monoxide, carbon dioxide or mixtures of both, wherein carbon dioxide is preferred.

Process conditions for the hydrogenation of carbon monoxide or mixtures of carbon monoxide and carbon dioxide are known perse, for example a low-pressure synthesis, a medium-pressure-synthesis and a high-pressure synthesis.
i) Low-pressure synthesis
The low-pressure synthesis is generally carried out at pressures between 50 and 100 bar. The temperature is generally 220 to 300° C. As a catalyst, generally a catalyst based on Cu, Zn and Al₂O₃ (e.g. CuO/ZnO/Al₂O₃) is used. The low-pressure synthesis is the most preferred synthesis for the preparation of methanol from carbon monoxide or from mixtures of carbon monoxide and carbon dioxide.
ii) Medium-pressure-synthesis
The medium-pressure-synthesis is generally carried out at pressures between 100 and 250 bar. The temperature is generally up to 300° C. As catalysts, generally a catalyst based on Zn/Cr₂O₃ or Zn—Cu catalysts are used.
iii) High-pressure synthesis The high-pressure-synthesis is generally carried out at pressures between 250 and 350 bar. The temperature is generally 320 to 380° C. As a catalysts, generally a catalyst based on zinc-chromium oxide is used. This process is less preferred for the production of methanol from carbon monoxide or from mixtures of carbon monoxide and carbon dioxide.

The current world energy system is still mainly based on the use of fossil fuels and, although the use of renewable energy sources has increased, it will continue in the medium and short term. The massive use of fossil fuels in industry and transport produce large amounts of CO₂ emissions. Since it is an object of the present invention to provide environmentally friendly ethanolamines, polyethylenimine and ammonia and an environmentally friendly process for making the same, it is preferred that methanol is prepared by reacting the hydrogen from step (a) with carbon dioxide in step (c) according to the process of the present invention.

In preferred embodiments, the carbon dioxide that is provided in step (c) is captured from industrial flue gases or from ambient air. All available capture technologies may be used.

An overview of commercial CO₂ capturing technologies is given in Koytsoumoa et al., The Journal of Supercritical Fluids, Volume 132, February 2018, Pages 3-16.

Capturing CO₂ is most cost-effective at point sources, such as large carbon-based energy facilities, industries with major CO₂ emissions (e.g. cement production, ammonia synthesis, steelmaking), natural gas processing, synthetic fuel plants and fossil fuel-based hydrogen production plants. Extracting CO₂ from air is possible, although the lower concentration of CO₂ in air compared to combustion sources complicates the engineering and makes the process therefore more expensive.

In some preferred embodiments, the carbon dioxide that is provided in step (b) is captured from industrial flue gases.

The main industrial sources of CO₂ are power plants based on burning of fossil fuels, oil refineries, biogas sweetening (e.g. fermentation) as well as the production of chemicals. Relevant chemical production processes are e.g. naphta cracking for C₁-C₄ olefins and C₆ aromatics as well as downstream chemicals such as especially ammonia and other CO₂-intensive products). Furthermore industrial paper, food, cement, mineral and iron and steel production can be named as examples.

In post combustion capture, the CO₂ is removed after combustion of the fossil fuel—this is the scheme that would apply to fossil-fuel power plants. CO₂ is captured from flue gases at power stations or other point sources. Absorption, or carbon scrubbing with amines is the dominant capture technology. It is the only carbon capture technology so far that has been used industrially. Suitable post carbon capture methods are for example absorption (chemical, physical), adsorption (chemical, physical), membrane processes, biological and cryogenic processes.

Pre-conversion capture means capturing CO₂ generated as an undesired co-product of an intermediate reaction of a conversion process. Some examples include the production of ammonia and coal gasification in power plants. In ammonia production, CO₂ that is co-produced with hydrogen during steam reforming must be removed before the ammonia synthesis can take place—absorption in monoethanolamine (MEA) and/or diethanolamine (DEA) is commonly used for these purposes. Similarly, in an integrated gasification combined cycle (IGCC) power plant, CO₂ must be separated from hydrogen. This is typically achieved using physical solvents such as selexol and rectisol. Note that, when applied in power plants, pre-conversion capture is also referred to as pre-combustion capture.

Oxy-fuel combustion technology involves the combustion of carbonaceous fuel in a stream of pure oxygen instead of air. Since the oxidant (O₂) is free of other components in the air (such as nitrogen), the CO₂ concentration in the flue gas will be very high, while the water vapor content can be easily removed.

CO₂ adsorbs to a MOF (Metal-organic framework) through physisorption or chemisorption based on the porosity and selectivity of the MOF leaving behind a CO₂ poor gas stream. The CO₂ is then stripped off the MOF using temperature swing adsorption (TSA) or pressure swing adsorption (PSA) so the MOF can be reused.

In some other preferred embodiments, the carbon dioxide that is provided in step (b) is captured from ambient air.

Direct air capture (DAC) is a process of capturing carbon dioxide (CO₂) directly from the ambient air and generating a concentrated stream of CO₂ for sequestration or utilization or production of carbon-neutral fuel. Carbon dioxide removal is achieved when ambient air makes contact with chemical media, typically an aqueous alkaline solvent or sorbents. These chemical media are subsequently stripped of CO₂ through the application of energy (namely heat), resulting in a CO₂ stream that can undergo dehydration and compression, while simultaneously regenerating the chemical media for reuse.

In Chen, Lackner et al., Angew. Chem. Int. Ed. 2020, 59, 6984-7006, “Sorbents for the Direct Capture of CO₂ from Ambient Air” describes major types of sorbents designed to capture CO₂ from ambient air categorized by the sorption mechanism: physisorption, chemisorption, and moisture-swing sorption.

In Kommalapati et al., Energy Technol. 2017, 5, 822-833, polyethylenimine applications in carbon dioxide capture and separation are described.

Dilute CO₂ can be efficiently separated using an anionic exchange polymer resin called Marathon MSA, which absorbs air CO₂ when dry, and releases it when exposed to moisture. A large part of the energy for the process is supplied by the latent heat of phase change of water. Other substances which can be used are metal-organic frameworks (or MOF's). Membrane separation of CO₂ rely on semi-permeable membranes.

In one embodiment of the present invention, the ethanolamines in a mixture or each of monoethanolamine, diethanolamine and triethanolamine, preferably in a mixture of monoethanolamine (MEA) and diethanolamine (DEA), and/or the polyethylenimine according to the present invention are employed in a process for capturing CO₂. The inventive ethanolamines in a mixture or each of monoethanolamine, diethanolamine and triethanolamine and the polyethylenimine do only add as little as possible, in a preferred embodiment nothing, to a CO₂ emission and do therefore as little as possible, preferably not, contribute to a CO₂ emission themselves.

In a further embodiment, the present invention therefore relates to the use of the ethanolamines, selected from monoethanolamine, diethanolamine, triethanolamine and mixtures thereof, preferably in a mixture of monoethanolamine (MEA) and diethanolamine (DEA), or the polyethylenimine according to the present invention as liquid and solid CO₂ absorbents in CO₂ capturing processes.

Suitable carbon capturing processes are mentioned above and known in the art.

In step (c) the carbon dioxide and hydrogen are reacted to form methanol.

Process conditions for the hydrogenation of carbon dioxide are known perse. Different process approaches are being developed for the synthesis of methanol by hydrogenation of CO₂: (1) heterogeneous catalysis, (2), homogeneous catalysis, (3) electrochemical, and (4) photocatalysis (see R. Guil-López, Materials 2019, 12, 3902; doi:10.3390/ma12233902). Preferably, the synthesis of methanol by hydrogenation of carbon dioxide is performed in the presence of a heterogeneous catalyst.

Generally, the methanol production is carried out in a synthesis converter, e.g. a fixed-bed, catalytic reactor.

The average temperature inside the reactor is generally in the range of 150 to 300° C. The average pressure inside the reactor is generally in the range of 50 to 150 bar (abs.).

An overview of suitable heterogeneous catalyst systems is given by Kristian Stangeland, Hailong Li & Zhixin Yu, Energy, Ecology and Environment volume 5, pages 272-285 (2020). Multi-component catalyst systems are required for this process. The interaction between components is essential for high activity and selectivity of CO₂-to-methanol catalysts. This has been demonstrated by numerous catalyst systems comprised of various metals (i.e., Cu, Pd, Ni) and metal oxides (i.e. Al₂O₃, ZnO, ZrO₂, In₂O₃). These complex systems can contain a mixture of metallic, alloy, and metal oxide phases. The most promising catalyst systems for large-scale industrial processes are currently Cu-based and In-based catalysts due to their superior catalytic performance. A suitable catalyst is for example copper-zinc-alumina.

By performing step (c), methanol is formed, CH₃, by reacting carbon oxides, preferably carbon dioxide, with the hydrogen from step (a). The deuterium content is even lower than corresponding to the distribution obtained by classical petrochemical routes.

### Step (d)

In step (d), methanol from step (c) is converted to ethylene and further to ethylene oxide.

Preferably, the ethylene oxide in step (d) is obtained by
(d1) a methanol-to-olefin process, wherein ethylene is obtained, followed by
(d2) epoxidation of ethylene.

### Step (d1)

In general, ethylene is produced from methanol in a methanol to olefin-process (MTO-process).

The MTO process is an acid catalyzed reaction. Preferred catalysts are zeolithes like zeolithes containing silica and alumina (e.g. ZSM-5) and silicon alumina phosphate zeolith-catalysts (SAPO) (e.g. SAPO-34).

This reaction is generally carried out at temperatures of from 300-600° C. The pressure is generally 0.1-0.3 MPa.

The process is preferably carried out in a fluidized catalytic reactor.

The ratio propylene to ethylene can be adjusted by choosing appropriate process conditions, and may vary from 0.77 in the ethylene production mode and 1.33 in the propylene production mode.

Examples for commercial MTO technology licensors are UOP (e.g. UOP Advanced MTO process), Energy Technology Co. Ltd. (DMTO process) and Sinopec (SMTO process).

More detailed descriptions can be found e.g. in “Ethylene” by Adam Chan, Nexant, TECH 2018-1, July 2018, p. 100-109.

### Step (d2)

In step (d2), the ethylene from step (d1) is converted to ethylene oxide.

The direct oxidation process is preferably performed in gas-phase, for example with oxygen or air, in the presence of a catalyst, preferably a silver catalyst, more preferably a silver catalyst supported on alumina.

The step (d2) is generally performed at a temperature of from 230 to 270° C. The pressure is preferably in the range of from 10 to 30 bar.

In a preferred embodiment, step (d2) is performed by gas-phase selective ethylene oxidation (ethylene epoxidation) that is typically performed in fixed-bed tubular reactors with supported Ag/Al₂O₃ catalysts at 230-270° C. and 10-30 bar.

Preferred catalysts for the process in step (d2) are silver-based catalysts like
supported Re/Cs/Ag/Al₂O₃ catalysts that operate preferably in excess C₂H₄/O₂; or
alkaline-metal (Na, Cs)-promoted supported Ag/Al₂O₃ catalysts that operate preferably in excess O₂/C₂H₄.

Oxides of Mo and S have been found to also promote the supported Re/Cs/Ag/Al₂O₃ system for EO formation. Therefore, the supported Re/Cs/Ag/Al₂O₃ system may additionally comprise oxides of Mo and/or S as promoters.

In addition, C₂H₄Cl₂ may also be added to deposit Cl on the catalyst, which acts as a promoter.

An example for a description can be found e.g. in “Ethylene Oxide” by Mia Monconduit and Karen Jobes, IHS Markit, Chemical Economics Handbook, 22 Dec. 2020, p. 14-16.

### Step (e)

In step (e), ammonia from step (b) is converted with ethylene oxide from step (d) to ethanolamines, selected from monoethanolamine, diethanolamine, triethanolamine and mixtures thereof in one or more steps.

The reaction product obtained in step (e) generally comprises monoethanolamine, diethanolamine and triethanolamine.

The preparation in step (e) is preferably performed in the presence of water, generally in a closed-cycle process with only minor fresh-water feed. However, it is also possible to prepare ethanolamines by reaction of ammonia and ethylene oxide in an anhydrous process. Anhydrous processes preferably employ a fixed-bed catalyst, for example an organic ion-exchange resin or thermally more stable acidic inorganic clays or zeolites.

In a preferred process, the reaction takes place in an aqueous phase, and the reactor pressure is usually sufficiently large to prevent vaporization of ammonia and ethylene oxide at the reaction temperature.

Ammonia concentrations in water are preferably between 50 and 100%.

The reaction pressure in the aqueous phase reaction is generally up to 160 bar, preferably 90 to 130 bar (abs).

The reaction temperature in the aqueous phase reaction is generally up to 150° C., preferably 40 to 130° C.

Generally, in the aqueous phase reaction an excess up to 40 mol of ammonia per mole of ethylene oxide is used.

Unconsumed ammonia and water are generally separated from the products in a distillation line downstream of the reactor and are recycled.

Product distribution of the three ethanolamines can be controlled by appropriate choice of the ammonia:ethylene oxide ratio.

Although the above reaction may be controlled by the stoichiometric ratio of the reactants ethylene oxide and ammonia, for obtaining monoethanolamine, usually a work-up by distillation is required to remove diethanolamine and triethanolamine.

In the preparation of polyethylenimine described in steps (f), (g) and (h) below, monoethanoamine is needed, which is generally separated from the ethanolamines which are generally obtained as mixtures of monoethanolamine, diethanolamine and triethanolamine in steps (a) to (e) of the process according to the present invention by distillation (see step (f) below).

However, in the case that the ethanolamines according to the present invention are employed in applications different from the preparation of polyethylenimines, a separation of monoethanolamine may not be necessary. In carbon capturing, for example, a mixture of monoethanolamine and diethanolamine may be employed and only triethanoamine has to be separated, generally by distillation.

Ethanolamines are a family of chemicals that work as surfactants and emulsifying ingredients in personal care products, cleaning products and industrial applications.

Further applications of the ethanolamines, selected from monoethanolamine, diethanolamine, triethanolamine and mixtures thereof according to the present invention are for example:

### Personal Care Products

Ethanolamines like MEA and especially TEA work as cleansing agents, or surfactants, in personal care products and cosmetics. In these types of products, ethanolamines help remove dirt and oil on skin by dissolving grease and blending other important ingredients. Because ethanolamines do not impart a strong odor, they are commonly ingredients in products like hair dye. Ethanolamines like MEA help adjust the pH of a product to keep it from degrading when stored in a container so it will last longer.

### Home Care and Industrial Cleaning Products

Ethanolamines such as MEA are common ingredients in cleaning products like floor and tile cleaners, as well as laundry detergents. As surfactants in these products, ethanolamines help remove dirt, grease and stains.

DEA is a common ingredient in industrial cleaning products, such as engine degreasers and industrial strength detergents, due to its ability to break down oil and grease.

### Industrial Applications

MEA acts as a plasticizing agent to help make plastic become pliable and soft. Chemical manufacturing plants use MEA to remove carbon dioxide from ammonia gas in the production of synthetic ammonia.

As a chemical intermediate, DEA is used in agrochemicals to make pesticides, where it helps increase a pesticide's ability to dissolve in water. In the production of wax, polish and coating products, DEA works as an emulsifier to help ingredients mix and help keep other materials from corroding.

Due to its emulsification properties, MEA and DEA also can be used in industrial applications, such as chemical manufacturing and gas treating. In gas treating processes for refineries and natural gas streams, MEA and DEA help remove contaminants from gasoline.

TEA is used as a surfactant in agrochemicals, to help pesticides disperse into crops, which then helps repel insects from the crops. As a petroleum demulsifier, TEA helps separate oil from water and other substances. In cement additives, TEA helps advance setting and/or hardening of cement. It is also a corrosion inhibitor in steel and zinc materials used in building and construction.

The present invention therefore further relates to the use of the inventive ethanolamines selected from monoethanolamine, diethanolamine, triethanolamine and mixtures thereof in any one of the applications mentioned above.

Carbon capturing agents, surfactants, emulsifying agents, cleansing agents, pH adjusting agents, plasticizing agents, gas sweetening agents, anticorrosion additives and cement additives comprising at least one ethanolamine selected from monoethanolamine, diethanolamine and triethanolamine according to the present invention.

An example for a description can be found e.g. in “Ethanolamines” by Mia Monconduit and Tison Keel, IHS Markit, Chemical Economics Handbook, 14 Feb. 2020, p. 19 20.

The present invention further relates to ethanolamines, selected from monoethanolamine, diethanolamine, triethanolamine and mixtures thereof, wherein the molar share of deuterium is ≤100 ppm, preferably in the range of from 10 to ≤98 ppm, more preferably in the range of from 10 to ≤95 ppm, most preferably in the range of from 10 to ≤90 ppm, based on the total hydrogen content.

The ethanolamines, selected from monoethanolamine, diethanolamine, triethanolamine and mixtures thereof according to the present invention are characterized by a low deuterium molar share.

Said ethanolamines, selected from monoethanolamine, diethanolamine, triethanolamine and mixtures thereof are preferably prepared by a process comprising at least step (a) according to the present invention. More preferably, said ethanolamines, selected from monoethanolamine, diethanolamine, triethanolamine and mixtures thereof are prepared by the of the present invention comprising steps (a) to (e).

The present invention further relates to a process for preparing polyethylenimine, wherein said process comprises:
(f) separating monoethanolamine from ethanolamines obtained in steps (a) to (e) of the process according to the present invention, or from ethanolamines according to the present invention;
(g) converting monoethanolamine to ethylenimine,
(h) polymerizing ethylenimine from step (g) to polyethylenimine.

### Step (f):

Step (f) comprises separating monoethanolamine from ethanolamines obtained in steps (a) to (e) of the process according to the present invention, or from ethanolamines according to the present invention.

Although reaction step (e) may be controlled by the stoichiometric ratio of the reactants ethylene oxide and ammonia, for obtaining monoethanolamine, usually separation is required to remove diethanolamine and triethanolamine. Said work-up is generally known in the art and is usually performed by distillation.

An example for a monoethanolamine production process description can be found e.g. in “Ethanolamines and Propanolamines” by Martin Ernst, Johann-Peter Melder, Franz Ingo Berger and Christian Koch, Ullmann's Encyclopedia of Industrial Chemistry, 2022, p. 4-6.

### Step (g):

In step (g), monoethanolamine is converted to ethylenimine (aziridine).

Suitable processes for the preparation of ethylenimine from monoethanolamine are generally known in the art.

A preferred commercial process is catalytic gas phase dehydration, see e.g. U.S. Pat. No. 4,841,061 for suitable examples of catalyst compositions and U.S. Pat. No. 4,966,980 for the respective ethylenimine gas phase process conditions.

In this process ethylenimine is prepared by dehydration of monoethanolamine at temperatures of generally 350 to 450° C., generally at reduced pressure of 30 to 500 mbar(abs).

The process is generally carried out in the presence of a catalyst with weak basic and acidic sites, which is for example based on Si—Cs—P or Si—Rb—P. An overview of this production technology and optimal catalyst composition with high yield (up to 80 mol %), selectivity and activity is found in Applied Catalysis A: General 221 (2001) 209-217; Acid-base catalysis: On the example of ethylenimine production; Hideaki Tsuneki; Nippon Shokubai Co. Ltd., Functions and Materials Research Laboratory, Suita 564-8512, Japan. The process is susceptible to catalyst deactivation by coking and sintering as well as loss of active components due to the high processing temperature despite low contact times. In order to improve catalyst lifetime a catalyst regeneration cycle with e.g. trimethylphosphate-treatment can be employed as described in Applied Catalysis A: General 331 (2007) 95-99; Deactivation and regeneration of ethylenimine production catalyst; Hideaki Tsuneki, Kimio Ariyoshi; Nippon Shokubai Co. Ltd., Functions and Materials Research Laboratory, Suita 564-8512, Japan

The catalytic gas phase dehydration process is usually carried out in the gas phase in a flow tube reactor.

The product mixture obtained is usually separated by quenching followed by multistage distillation giving high purity ethylenimine, and unreacted monoethanolamine is fed back into the reactor.

Another preferred commercial process is liquid phase dehydration via the Wenker process as initially described in H. Wenker, J. Am. Chem. Soc. 57 (1935) 2328. This technology is generally a two-step process reacting monoethanolamine generally with sulfuric acid to 2-aminoethyl hydrogensulfate as an intermediate product. By susequent addition of usually sodium hydroxide the dehydration is achieved generally under pressure and elevated temperatures as described in H. Kindler, W. Sanne, R. Sinn, A. Wittwer, Chem. Ing. Tech. 37 (1965) 400 and DE 1302658, 1971 (R. Sinn, W. Sanne, H. Kindler; BASF Aktiengesellschaft).

The liquid phase ethylenimine process can be generally carried out in batch or continuous mode usually with very high yields of 85-90 mol % giving high purity ethylenimine after distillation with excellent properties required e.g. for high molecular weight polyethylenimines and other derivatives.

### Step (h)

In step (h) ethylenimine from step (g) is polymerized to polyethylenimine.

Suitable processes for the preparation of polyethylenimine by polymerization of ethylenimine (aziridine) are known by a person skilled in the art.

Polyethylenimine is preferably prepared by cationic ring opening polymerization of ethylenimine in the presence of Broensted acids, Lewis acids, haloalkanes or carbon dioxide. Examples are given in U.S. Pat. Nos. 2,182,306 and 3,203,910 as well as US 2001/0039318.

As a further reference with further examples for polyethylene synthesis it is referred to “Aziridines and azetidines: building blocks for polyamines by anionic and cationic ring-opening polymerization” Gleede, T.; Reisman, L.; Rieger, E.; Mbarushimana, P. C.; Rupar, P. A.; Wurm, F. R.; Polymer Chemistry 2019, 10, 3257.

The polymerization may be carried out for example in a batch process in which water and 1,2-dichloroethane as catalyst are placed in a reaction vessel, the mixture is heated to a temperature of from 70 to 100° C. and ethylenimine is continuously added with stirring of the reaction mixture.

The polyethylenimines obtained are generally branched or hyperbranched polyethylenimines.

The polyethylenimine obtained has preferably a weight average molecular weight M_{w} in the range of from 500 to 2,000,000 g/mol, preferably in the range of from 500 to 100,000 g/mol.

The degree of branching of the polyethylenimines is preferably in the range of from 0.45 to 0.75, more preferably 0.5 to 0.7, most preferably 0.55 to 0.7, determined by ¹³C NMR spectroscopy in D₂O. The degree of branching is calculated as (D+7)/(D+T+L). In this formula, D refers to the dendritic (or tertiary) amin groups, L (linear) refers to the secondary amino groups and L (linear) to the primary amino groups.

The inventive polyethylenimine is characterized by a low molar share of deuterium, which is ≤110 ppm, preferably in the range of from 10 to ≤105 ppm, more preferably in the range of from 10 to ≤95 ppm, most preferably in the range of from 10 to ≤92 ppm, based on the total hydrogen content.

The present invention further relates to polyethylenimine, wherein the molar share of deuterium is ≤110 ppm, preferably in the range of from 10 to ≤105 ppm, more preferably in the range of from 10 to ≤95 ppm, most preferably in the range of from 10 to ≤92 ppm, based on the total hydrogen content.

Said polyethylenimines are preferably prepared by a process comprising at least step (a) according to the present invention. More preferably, said polyethylenimines are prepared by the process of the present invention comprising steps (a) to (h).

The polyethylenimines according to the present invention are characterized by a low deuterium molar share. They display a deuterium share different from petrochemically made polymers and ethanolamine made by a petrochemical process, i.e. based on fossil energy.

The key feature of the ethanolamines, selected from monoethanolamine, diethanolamine, triethanolamine and mixtures thereof and the polyethylenimine of the present invention is a low deuterium molar share. Said low deuterium molar share is mainly introduced by step (a) of the process according to the present invention. Therefore, also the ammonia, prepared by reacting the hydrogen from step (a) with nitrogen is characterized by a low deuterium molar share.

The present invention therefore further relates to a process for making ammonia wherein said process comprises the following steps:
(a) providing hydrogen with a molar share of deuterium (deuterium content) below 90 ppm, based on the total hydrogen content, by electrolysis based on electrical power generated at least in part from non-fossil energy, preferably renewable recources,
(b) reacting the hydrogen from step (a) with nitrogen to form ammonia.

Steps (a) and (b) are the same steps as described above.

The present invention further relates to ammonia wherein the molar share of deuterium is ≤100 ppm, preferably in the range of from 10 to ≤95 ppm, more preferably in the range of from 10 to ≤90 ppm, most preferably in the range of from 10 to ≤80 ppm, based on the total hydrogen content.

With the present invention environmentally friendly ethanolamines, selected from monoethanolamine, diethanolamine, triethanolamine and mixtures thereof, polyethylenimine and ammonia and an environmentally friendly process for making the same, wherein said process uses as little fossil energy as possible, are provided.

It has been found that said environmentally friendly prepared compounds, i.e. ethanolamines, selected from monoethanolamine, diethanolamine, triethanolamine and mixtures thereof, polyethylenimine and ammonia are characterized by a specifically low deuterium molar share.

As mentioned above, it is important that the origin of the hydrogen and downstream compounds obtained by clean energy can be tracked in a reliable way.

Today, the majority of hydrogen is produced from fossil fuels by steam reforming of natural gas and other light hydrocarbons, partial oxidation of heavier hydrocarbons, and coal gasification.

However, up to now, there was no possibility to distinguish the hydrogen obtained by by steam reforming, partial oxidation and coal gasification, i.e. by fossil resources, from hydrogen obtained by electrolysis. As discussed above, hydrogen obtained by electrolysis is preferably obtained by using non-fossil energy sources. It is expected that the electrification (power generation) of fossil sources will be fully replaced by the generation of power by non-fossil resources in the near future.

The inventors therefore found a way for tracing the origin of hydrogen and downstream products of hydrogen, preferably ethanolamines, selected from monoethanolamine, diethanolamine, triethanolamine and mixtures thereof, polyethylenimine, ammonia and hydrogen via the deuterium molar share of said compounds. The downstram products based on hydrogen, preferably ethanolamines, selected from monoethanolamine, diethanolamine, triethanolamine and mixtures thereof, polyethylenimine, ammonia based on hydrogen obtained by electrolysis and hydrogen itself can be distinguished by its deuterium molar share from ethanolamines, selected from monoethanolamine, diethanolamine, triethanolamine and mixtures thereof, polyethylenimine, ammonia and hydrogen prepared by processes based on fossil energy, i.e. made by petrochemical processes.

Furthermore by using carbon oxides such as carbon monoxide and preferably carbon dioxide together with hydrogen instead of petrochemical synthesis gas in the subsequent synthesis routes for ethanolamines, selected from monoethanolamine, diethanolamine, triethanolamine and mixtures thereof, polyethylenimine and ammonia the molar share of deuterium in these compounds was found to be uniquely low with excellent traceability.

The present invention therefore relates to the use of the molar share of deuterium in hydrogen and downstream compounds based on hydrogen for tracing the origin, especially the energetic origin, of the hydrogen and downstream compounds based on hydrogen, wherein the compounds are preferably ethanolamines, selected from monoethanolamine, diethanolamine, triethanolamine and mixtures thereof, polyethylenimine or ammonia.

The present invention further relates to a process for tracing the origin, especially the energetic origin, of hydrogen and downstream compounds based on hydrogen by determining the molar share of deuterium in hydrogen and said downstream compounds based on hydrogen, wherein the compounds are preferably ethanolamines, selected from monoethanolamine, diethanolamine, triethanolamine and mixtures thereof, polyethylenimine or ammonia.

Downstream products based on hydrogen are generally products prepared by using hydrogen, preferably ethanolamines, selected from monoethanolamine, diethanolamine, triethanolamine and mixtures thereof, polyethylenimine, ammonia as well as methanol. The preparation of the downstream products as well as other downstream products based on hydrogen are known in the art.

Tracing is in the meaning of the present invention synonymous with tracking.

The origin is in the meaning of the present invention the preparation method of the hydrogen employed, especially electrolysis and/or the energetic origin, i.e. non-fossil energy sources. As mentioned above, it is expected that the electrification (power generation) of fossil sources will be fully replaced by the generation of power by non-fossil resources in the near future. Hydrogen made by electrolysis is in this case hydrogen of non-fossil origin. Examples for non-fossil power sources are mentioned above.

The inventive process for tracing the origin, especially the energetic origin, of hydrogen and downstream compounds mentioned above may be employed as a single tracing (tracking) method or in combination with further tracing (tracking) methods.

Suitable deuterium molar shares of the compounds based on hydrogen, especially ethanolamines, selected from monoethanolamine, diethanolamine, triethanolamine and mixtures thereof, polyethylenimine or ammonia, and the hydrogen itself obtained based on hydrogen made by electrolysis, especially obtained by the process of the present invention, are mentioned in the present application.

Due to their polyamine structure, polyethylenimine (PEI) polymers are capable of protonation, chelation, and reaction, making them very attractive in a variety of applications.

As mentioned above, PEI is useful in reducing greenhouse gas emissions in CO₂ capturing, for example in form of gas separation membranes for CO₂ capturing as well as for H₂ purification from gas mixtures such as CO₂/N₂ and H₂/N₂.

Further examples for typical applications of PEI are:
Wet-strength additive in the paper industry to improve the strength of paper products.
Use in detergents and cosmetics
Adhesion promoters for printing inks and adhesives
Primers in coating applications to improve adhesion to various substrates such as glass, wood, plastics and metal
Flocculants to precipitate colloidal particles from water
Removal of heavy-metal ions from sewage (e.g. Cu²⁺, Pb²⁺, Cr⁶⁺, Cd²⁺)
Green blowing agents for polyurethanes
Chelating agent with the ability to complex metal ions such as zinc and zirconium
Antimicrobial coatings: PEI can be used as an antimicrobial coating for surfaces. It has been shown to have activity against a wide range of microorganisms, including bacteria, viruses, and fungi
Textile industry: PEI can be used as a crosslinking agent in the textile industry to improve the durability and strength of fabrics
Food packaging: PEI can be used as a coating for food packaging materials to improve their barrier properties and extend the shelf life of food products.

Further applications of PEI are in the biological and medical fields:
Gene transfection agents for biomedical applications
Mucosal adjuvant for various vaccines
Design of immobilized enzyme biocatalysts
Use in laboratory biology, especially tissue culture
Attachment promoter for weakly anchoring cells in cell culture
Gene delivery: PEI can be used as a gene delivery agent in gene therapy. It has been shown to efficiently deliver plasmid DNA into cells and can be used for both in vitro and in vivo applications
Protein purification: PEI can also be used as a purification agent for proteins. It can selectively bind to proteins and is particularly useful for purifying negatively charged proteins.
Biomedical implants: PEI can be used as a coating for biomedical implants to improve their biocompatibility and reduce the risk of rejection by the body.

The present invention therefore further relates to the use of the inventive polyethylenimine in any one of the applications mentioned above.

The present invention further relates to CO₂ capturing agents, gas separation membranes especially for CO₂ capturing and for H₂ purification from gas mixtures such as CO₂/N₂ and H₂/N₂, wet-strength additive in the paper industry, detergents, cosmetics, adhesion promoters especially for printing inks and adhesives, primers in coating applications, flocculants to precipitate colloidal particles from water, chelating agents especially for heavy-metal ions in sewage, green blowing agents for polyurethanes, antimicrobial coatings, crosslinking agents in the textile industry, coatings for food packaging materials, gene transfection agents for biomedical applications, mucosal adjuvant for various vaccines, immobilized enzyme biocatalysts, attachment promoters for weakly anchoring cells in cell culture, gene delivery agents in gene therapy, purification agents for proteins, coatings for biomedical implants comprising or consisting of at least one polyethylenimine according to the present invention.

The invention is further illustrated by working examples.

### EXAMPLES

### Experimental Results—Overview

TABLE 1

| | Electrolyser type | | | | | | |
|---|---|---|---|---|---|---|---|
| | Example I) | Example II) | Example III) | Example IV) | | | |
| | PEM- | PEM | Alkaline | Alkaline | Example V) | Example VI) | Example VII) |
| | electrolyser | electrolyser | electrolyser | electrolyser | AEM | AEM | Solid oxide |
| Water- | VSMOW/155.76 | Selected* | VSMOV/155.76 | Selected* | VSMOW/155.76 | Selected* | Selected* |
| type/deuterium | ppm D | surface | ppm D | surface | ppm D | surface | surface |
| content | | water/139 | | water/139 | | water/139 | water/139 |
| | | ppm D | | ppm D | | ppm D | ppm D |
| Current density | 1.35 | 1.35 | 0.8 | 0.8 | 1.6 | 1.6 | 0.78 |
| [A/cm2] | | | | | | | |
| Applied voltage | 1.9 | 1.9 | 2.15 | 2.15 | 1.9 | 1.9 | 1.35 |
| [V] | | | | | | | |
| Operating | 68 | 68 | 81 | 81 | 45 | 45 | 710 |
| temperature [° C.] | | | | | | | |
| Cell pressure [bar] | 23 | 23 | 25 | 25 | 35 | 35 | 1.3 |
| Voltage efficiency | 57 | 57 | 53 | 53 | 66 | 66 | 81 |
| [%] | | | | | | | |
| Electrical | 52 | 52 | 48.5 | 48.6 | 69 | 69 | 47 |
| efficiency | | | | | | | |
| [kWh/kg H2] | | | | | | | |
| Deuterium content | 86 | 77 | 95 | 87 | 74 | 66 | 38 |
| in hydrogen [ppm] | | | | | | | |

*Isar river surface water (Munich) collected in winter season January 2021.

### I Hydrogen Production

Experimental Setup and Method: Electrolysis cell design and Hydrogen production conditions

### 1) Polymer Electrolyte Membrane/Proton Exchange Membrane Electrolysis (PEM)

### Electrolysis Cell

Water electrolysis was conducted with a circular commercial PEM electrolysis cell (model ZE 200, Sylatech Analysetechnik GmbH, 0.007 m² active area). The cell stack was sealed with O-rings and wrapped with heat insulation fabric for isothermal operating conditions. A Nafion® 117 standard membrane (supplier DuPont, dry thickness 180 microns) was assembled by HIAT GmbH with catalytic active coating materials iridium (19 g/m²) and platinum (8 g/m²). Water distribution at the anode half-cell was realized with a titanium mesh. Before the polymer electrolyte membrane, a porous transport layer with sintered titanium fiber material is used for controlled flow while a porous graphite plate was situated on the cathode-side.

### Experimental Conditions

Water with controlled temperature was supplied at constant flow of 9.5 g/h to the anode compartment. The cell pressure on cathode and anode side was controlled with PC valves. Experimental conditions such as temperature and pressure settings see table.

The evolved hydrogen and oxygen gas in the half-cells was separated in a two-step separator setup with an intermediate condenser cooled with 20° C. cooling water. Water condensate flowed back to the separator tank. Anodic cell-water was re-cycled, whereas on the cathode the separated water was drained. Remaining gas moisture is separated by desiccant dryers, ensuring complete separation of moisture in the electrolysis gas flow. Small gas samples of the dehydrated cathodic hydrogen gas were taken from the continous flow with an auto-sampler at regular intervals to conduct hydrogen gas analytics described below.

### 2) Alkaline Electrolysis Cell (AEC)

### Electrolysis Cell

Alkaline water electrolysis was conducted with a circular AEC electrolysis cell (model Electro MP Cell, supplier ElectroCell Europe A/S, 0.01 m² electrode area). As electrodes Nickel 2.4068 material was used and separated by a commercial standard Zirfon Perl UTP 500 membrane (open mesh polyphenylene sulfide fabric symmetrically coated with a mixture polymer/zirconium oxide; thickness 500 microns; 0.023 m² active area; supplier Agfa-Gevaert N.V.) in zero-gap cell configuration.

### Experimental Conditions

Alkaline water (32 wt % potassium hydroxide, technical standard grade) with controlled temperature was supplied at constant flow of 27.8 kg/h to the anode and cathode compartment. The cell pressure on cathode and anode side was equalized via PC valve control. Experimental conditions such as temperature and pressure settings see table. The evolved hydrogen and oxygen gas in the half-cells was separated in a two-step separator setup with an intermediate condenser cooled with 20° C. cooling water. Water condensate flowed back to the separator tank. Anodic and cathodic cell-water was recycled. Remaining gas moisture is separated by desiccant dryers, ensuring complete separation of moisture in the electrolysis gas flow. Small gas samples of the dehydrated cathodic hydrogen gas were taken from the continuous flow with an auto-sampler at regular intervals to conduct hydrogen gas analytics described below.

### 3) Anion Exchange Membrane/Alkaline Electrolyte Membrane Electrolysis (AEM)

### Electrolysis Cell

The AEM experiments were executed in a commercial, fully automated 2.4 kW EL 4.0 cell supplied by Enapter GmbH, 10117 Berlin and a 1 wt % potassium hydroxide (standard grade) electrolyte solution.

### Test Station

As recommended by the supplier the EL 4.0 electrolyzer is run with a 1 wt % potassium hydroxide (technical standard grade) solution, hydrogen production at operating conditions (experimental conditions such as temperature and pressure settings see table) was 480 I/h at approx. 400 ml water consumption. The evolved hydrogen gas was treated with desiccant dryers, ensuring complete separation of moisture in the electrolysis gas flow to yield <0.03 wt % water in the hydrogen gas stream. Small gas samples of the dehydrated cathodic hydrogen gas were taken from the continous flow with an auto-sampler at regular intervals to conduct hydrogen gas analytics described below.

### 4) Solid Oxide Electrolysis Cell (SOE)

Solid Oxide cell stacks from Elcogen-Elco Stack (Elcogen O Y, Vantaa 01510 Finland), were used and a E3000 unit was operated in reverse mode at 700° C. and 35A electrolyzing current; Anode functional composition due to the supplier is NiO/YSZ. Cathode is of LSC type [La(Sr)CoO3]: The principle is also described in Novel high-performance solid oxide fuel cells with bulk ionic conductance dominated thin-film electrolytes—ScienceDirect; D. Stöver et al, Journal of Power Sources; Volume 218, 15 Nov. 2012, Pages 157-162.

The hydrogen stream was used with no further purification/dryer. Remaining gas moisture is separated by desiccant dryers, ensuring complete separation of moisture in the electrolysis gas flow. Small gas samples of the dehydrated cathodic hydrogen gas were taken from the continuous flow with an auto-sampler at regular intervals to conduct hydrogen gas analytics described below.

The results of the hydrogen production are shown in table 1.

Experimental Setup and Method: “D content (Deuterium content) in samples”

The following method descriptions apply for determination of the molar share of deuterium based on the total hydrogen content (Deuterium content) of gas and liquid samples. The isotopic H/D-share analysis is based on mass spectroscopy. Two different methods are used: method A for gas samples and method B for liquid samples.

For determination of the “D content in gas and liquid samples” it is of crucial importance not to contaminate the samples e.g. with ambient humidity or other ambient components containing hydrogen or deuterium. Therefore gas-tight materials and sealings must be used with clean sample containers to avoid any cross-contamination. Therefore, before filling and sealing a sample container it must be flushed at least 20 times the sample container volume with the gas or liquid stream to be analyzed. The same is valid for the experimental setup of the gas sampler and mass spectrometer. Utmost care must be taken to avoid cross-contamination e.g. via condensation of humidity. The analytical setup from sampling to mass spectrometry is validated with known reference samples.

### Method A) Gas Samples

Total Deuterium from HD and D₂ in hydrogen gas samples was determined via ultrahigh resolution quadrapole mass spectrometry using a Hiden DLS-20 (Hiden Analytical Ltd., Warrington, Cheshire, UK) analyzer setup. The general method setup is described in C. C. Klepper, T. M. Biewer, U. Kruezi, S. Vartanian, D. Douai, D. L. Hillis, C. Marcus, Extending helium partial pressure measurement technology to JET DTE2 and ITER; Rev. Sci. Instrum., 87 (11) (2016); doi: 10.1063/1.4963713. For the hydrogen gas samples the threshold ionization mass spectrometry mode (TIMS) was used as described in S. Davies, J. A. Rees, D. L. Seymour; Threshold ionisation mass spectrometry (TIMS); A complementary quantitative technique to conventional mass resolved mass spectrometry; Vacuum, 101 (2014), pp. 416-422; doi: 10.1016/j.vacuum.2013.06.004. Sensitivity is +/−1 ppm.

### Method B) Liquid Samples

Analysis of liquid samples (ammonia, monoethanolamine (MEA) and polyethylenimine (PEI)) was executed via isotope ratio monitoring gas chromatography/mass spectrometry (IRMS). Therefore a DELTA V PLUS CF-IRMS mass spectrometer was used. This mass spectrometer with magnetic sector with continuous flux DELTA V PLUS CF-IRMS is used to measure the isotopic ratio of 2 H/1 H.

Measurement of D/H in a continuous He-flow mode needs the complete removal of low energy 4 He+ ions from the HD+ ion beam at m/z 3). The method is described in RAPID COMMUNICATIONS IN MASS SPECTROMETRY Rapid Commun. Mass Spectrom. 13, 1226-1230 (1999), W. A. Brandt et al. Sensitivity is within +/−3 ppm.

II Production of Ammonia, ethylene oxide (EO), ethanolamines and polyethylenimine (PEI)

Ammonia, ethylene oxide as well as ethanolamines as pure components monoethanolamine (MEA), diethanolamine (DEA) or triethanolamine (TEA) or mixtures of MEA, DEA and/or TEA are prepared with catalytic synthesis processes in industrial scale as described above.

By using non-fossil based energy for electrolytic hydrogen in both the ammonia and methanol synthesis at the root of this value chain, the only material hydrogen in the downstream resulting EO, MEA, DEA and TEA is actually originating from the electrolytic hydrogen with a low deuterium content. Especially for ammonia this is a big difference since the fossil-based production process relies on synthesis gas made from fossil natural gas and additionally large quantities of steam. This steam reforming step is not required for the non-fossil ammonia route based on pure hydrogen and nitrogen.

In all of the large-scale commercial production processes in question using non-fossil hydrogen no significant additional amounts of hydrogen-species are introduced in the “input/output” process material balance. This is required in order to minimize output of undesired wastewater or other liquid and gaseous emission streams and to ensure high yields with purity according to the product specification.

If ambient air used for direct oxidation of ethylene in EO production the ambient moisture does not participate in the catalytic reaction and is therefore purged together with nitrogen and other inert gases. The EO product as such thus consists practically only of material hydrogen originating from the ethylene. Ethylene made via MTO or similar processes from methanol from hydrogen and carbon oxides has almost the same deuterium concentration as the hydrogen source.

In case water is used as processing aid for stripping/scrubbing/cleaning or quenching/condensing of process-internal streams in e.g. the ammonia, methanol, ethylene oxide and/or ethanolamine synthesis steps this water is always run in an almost completely closed cycle with minimum purge ensuring insignificant cross-contamination with deuterium.

If hydrogen-species are introduced in the continuous industrial processes these feed streams are always minor compared to the output stream of the desired product, meaning at least 2 orders of magnitude smaller or in other words <1% of the output stream.

Therefore a worst-case factor 1.01 calculative increase of the deuterium content was used each for the ethylene (MTO based), ammonia and ethanolamine process steps.

For ethylenimine (EI) synthesis two different routes are commercially relevant as described. The liquid phase dehydration “Wenker process” is carried out in aqueous dilution with fresh water as well as sulfuric acid and caustic in molar quantities. Therefore the “acidic N-hydrogen” of the ethylenimine is practically completely exchanged with the deuterium content of the aqueous feed mix. This means that ⅕ of the molar share of deuterium can be assumed to have the VSMOV baseline concentration (155.76 ppm) but the remaining ⅘ hydrogen species maintain the low deuterium content of the ethanolamine source due to the strong nature of the C—H bond. Therefore the formula D-content (EI)=⅕ *D-content (VSMOV)+⅘ *D-content (MEA) is used. The catalytic gas-phase dehydration process does not introduce any significant amounts of hydrogen species apart from the MEA raw material and produces >98% concentrated ethylenimine monomer in the final sequence of distillation workup steps.

As with the upstream catalytic processes any bleeding of hydrogen species from e.g. partially acidic catalysts are not relevant in the material mass balance with industrially relevant high space-time yields.

However, ethylenimine itself is not a relevant commercial final product due to its hazardous toxicity and extreme reactivity. Polyethylenimine (PEI) products are made by cationic ring-opening polymerization of ethylenimine (EI) in aqueous solution with initiators as described above and fresh water in order to achieve removal of the strong exothermic heat of polymerization. Polymerization without significant dilution of water cannot be safely controlled in large scale. Industrially common PEI products are therefore usually polymerized in batch oder semi-batch mode with temperature control at e.g. 30 or 50% aqueous final concentration. This means that the “acidic N-hydrogen” from ethylenimine is practically completely exchanged with the deuterium content of the fresh water used in the polymerization step. Since this H-D exchange on the “acidic N-hydrogen” can only happen once it follows that the result for the deuterium content is practically identical: Both polyethylenimines based on either the liquid or the gas-phase dehydration EI monomer process have the same deuterium content approximated by the formula D-content (PEI)=⅕ *D-content (VSMOV)+⅘ *D-content (MEA).

In table 2, the deuterium (D) content in hydrogen, ammonia, monoethanolamine (MEA) and polyethylenimine (PEI) based on fossil resources (FR's) (comparative examples) and non-fossil resources (NFR's) (inventive examples) is shown. The products from FR's (comparative examples) are conventional petrochemical products from BASF SE. The deuterium contents of products from NFR's are calculated as described above using the D content of hydrogen from examples I) to VI) (see table 1) as starting materials for both the ammonia and ethylene synthesis routes.

Comparative examples: Products based on fossil resources

| | D content [ppm] | D content [ppm] | D content [ppm] | D content [ppm] |
|---|---|---|---|---|
| Comparative | in hydrogen | in ammonia | in MEA from | in PEI from |
| Example | from FR's | from FR's | FR's | FR's |
| | 131 | 124 | 126 | 120 |

Inventive examples: Products based on non-fossil resources

| | D content [ppm] | D content [ppm] | D content [ppm] | D content [ppm] |
|---|---|---|---|---|
| Inventive | in hydrogen | in ammonia | in MEA from | in PEI from |
| Examples | from NFR's | from NFR's | NFR's | NFR's |
| AI) | 86 | 87 | 89 | 102 |
| AII) | 77 | 78 | 79 | 95 |
| AIII) | 95 | 96 | 98 | 109 |
| AIV) | 87 | 88 | 90 | 103 |
| AV) | 74 | 75 | 76 | 92 |
| AVI) | 66 | 67 | 68 | 86 |
| AVII) | 38 | 38 | 39 | 62 |

The examples based on NFR's show a significantly lower D content and can be clearly distinguished from conventional products based on FR's.

1.-17. (canceled)

18. Ethanolamines, selected from monoethanolamine, diethanolamine, triethanolamine and mixtures thereof, wherein the molar share of deuterium is ≤100 ppm, based on the total hydrogen content.

19. A process for making ethanolamines according to claim 18, selected from monoethanolamine, diethanolamine, triethanolamine and mixtures thereof, wherein said process comprises the following steps:
(a) providing hydrogen with a molar share of deuterium ≤100 ppm, based on the total hydrogen content, by electrolysis based on electrical power generated at least in part from non-fossil energy,
(b) reacting the hydrogen from step (a) with nitrogen to form ammonia,
(c) reacting the hydrogen from step (a) with carbon oxides, preferably carbon dioxide to form methanol,
(d) converting the methanol from step (c) to ethylene and further to ethylene oxide,
(e) converting the ammonia from step (b) with ethylene oxide from step (d) to ethanolamines in one or more steps.

20. The process according to claim 19 wherein the electrical power is generated at least in part from wind power, solar energy, hydroelectricity, geothermal energy, ambient heat captured by heat pumps, bioenergy, the renewable part of waste, or nuclear power.

21. The process according to claim 19, wherein step (a) is a water electrolysis.

22. The process according to claim 19, wherein carbon dioxide is employed in step (c).

23. The process according to claim 19 wherein the ethylene oxide in step (d) is obtained by:
(d1) a methanol-to-olefin process, wherein ethylene is obtained; and
(d2) epoxidation of ethylene.

24. The process according to claim 19, wherein ammonia from step (b) and ethylene oxide from step (d) are reacted in one or more steps to a reaction product comprising monoethanolamine, diethanolamine and triethanolamine.

25. Polyethylenimine having a molar share of deuterium of ≤110 ppm, based on the total hydrogen content.

26. A process for preparing polyethylenimine having a molar share of deuterium of ≤110 ppm based on the total hydrogen content, wherein said process comprises:
(f) separating monoethanolamine from ethanolamines obtained in steps (a) to (e) of the process according to claim 19;
(g) converting monoethanolamine to ethylenimine,
(h) polymerizing ethylenimine from step (g) to polyethylenimine.

27. The process according to claim 26, wherein step (g) is carried out in gas phase or in liquid phase.

28. Ammonia wherein the molar share of deuterium is ≤100 ppm, preferably in the range of from 10 to ≤95 ppm, more preferably in the range of from 10 to ≤90 ppm, most preferably in the range of from 10 to ≤80 ppm, based on the total hydrogen content.

29. A process for making ammonia according to claim 28 comprising:
(a) providing hydrogen with a molar share of deuterium ≤100 ppm, based on the total hydrogen content, by electrolysis based on electrical power generated at least in part from non-fossil energy; and
(b) reacting the hydrogen from step (a) with nitrogen to form ammonia.

30. A method of using a molar share of deuterium in hydrogen and downstream compounds based on hydrogen, comprising:
tracing an origin of preparation of hydrogen and downstream compounds based on the hydrogen; wherein the compounds are at least one of ammonia and an ethanolamine including monoethanolamine, diethanolamine, triethanolamine, polyethylenimine, and mixtures thereof.

31. A process for tracing the origin of preparation of hydrogen and downstream compounds based on hydrogen by determining the molar share of deuterium in in hydrogen and said downstream compounds based on hydrogen, wherein the compounds are preferably ethanolamines, selected from monoethanolamine, diethanolamine, triethanolamine and mixtures thereof, polyethylenimine or ammonia.

32. A CO2 capturing agent, gas separation membranes, wet-strength additive in the paper industry, detergents, cosmetics, adhesion promoters for printing inks and adhesives, primers in coating applications, flocculants to precipitate colloidal particles from water, chelating agents for heavy-metal ions in sewage, green blowing agents for polyurethanes, antimicrobial coatings, crosslinking agents in the textile industry, coatings for food packaging materials, gene transfection agents for biomedical applications, mucosal adjuvant for various vaccines, immobilized enzyme biocatalysts, attachment promoters for weakly anchoring cells in cell culture, gene delivery agents in gene therapy, purification agents for proteins or coatings for biomedical implants comprising the polyethylenimine according to claim 25.
